(19) United States
(12) Patent Application Publication
(10) Pub. No.: US 2008/0038776 A1
(43) Pub. Date: Feb. 14, 2008

(54) Method for Preparing a Macromolecule of Bacterial Origin, Macromolecule thus Obtained, and Use of the said Molecule in the Prevention and Treatment of Inflammatory Rheumatic Disease

(75) Inventors: Marie-Benedicte ROMOND, Lille (FR); Marie-Francoise ODOU-PARIS, Godewaersvelde (FR); Elisabeth SINGER, Lille (FR)

Correspondence Address: McLELAND PATENT LAW OFFICE, P.L.L.C., 11320 RANDOM HILLS ROAD, SUITE 250, FAIRFAX, VA, 22030, US

(73) Assignee: UNIVERSITE DU DROIT ET DE LA SANTE DE LILLE, Lille (FR)

(21) Appl. No.: 11/577,307

(22) PCT Filed: Oct. 14, 2005
(86) PCT No.: PCT/FR05/02554

§ 371 Date: Aug. 24, 2007

(30) Foreign Application Priority Data
Oct. 14, 2004 (FR) 0410886

(51) Int. Cl. C12P 21/06; C12P 19/62

(52) U.S. Cl. 435/69.1; 435/76

(57) ABSTRACT

The present invention relates to a method for the preparation of a macromolecule produced by bacteria of the Bifidobacterium breve I-2219 strain and the use of the said macromolecule for the preparation of medicines designed for the prevention and treatment of inflammatory rheumatic disease.

The present invention relates to a method for the preparation of a macromolecule of bacterial origin, the macromolecule thus obtained and the use of the said molecule for the prevention and treatment of inflammatory rheumatic disease.

A common denominator in numerous inflammatory rheumatic diseases such as rheumatoid polyarthritis, ankylosing spondylitis or post-infectious rheumatisms is the presence of bacteria or bacterial signals (DNA, RNA, peptidoglycan polysaccharides) in synovial fluid (“RT-PCR Analysis of Bacterial rRNA for Detection and Characterization of Bacterial Species in Arthritis Synovial Tissue”—Kempsell K. E., Cox C. J., Hurle M., Wong A., Wilkie S., Zanders E. D., Gaston J. S., Crowe J. S. In Infect. Immun. 2000, 68(10): 6012-26; “Investigation of Infectious Agents Associated with Arthritis by RT-PCR of Bacterial rRNA”, Cox C. J., Kempsell K. E., Gaston J. S. in Arthritis Res. Ther. 2003, 5(1):R1-8). These bacteria come for a large part from the digestive tract. They cross the intestinal barrier (bacterial translocation) and are transported by macrophages and dendritic cells to synovial sites. Since the joints of healthy subjects are usually sterile and devoid of bacterial residues, the presence of bacteria and their components in inflammatory synovia is considered to contribute to rheumatic disease.

Current treatments address the effects of rheumatic disease states and aim to reduce the inflammatory reaction, either by blocking pro-inflammatory cytokines (infliximab, enbrel, anakinra), or by reducing the renewal of immune cells responsible for the production of pro-inflammatory cytokines (leflunomide). However, such treatments have undesirable side effects such as infections (opportunistic infections, tuberculosis), anti-DNA antibodies and lupus, the risk of demyelinization of the central nervous system, neutropenia, and congestive heart failure.

The antibiotherapy applied to patients presenting a rheumatic disease state lowers the number of certain bacteria of the intestinal flora and contributes to reduce inflammation; however, this method of treatment cannot be used in the long term in view of the risk of emergence of resistant bacteria.

The present invention allows attenuating the disadvantages of the current methods for treating inflammatory rheumatic disease. The goal of the present invention is to propose another approach in the treatment of inflammatory rheumatic disease, based on the use of a macromolecule of bacterial origin capable of regulating the intestinal flora and bacterial translocation and hence, reducing the inflammatory reaction that is associated with inflammatory rheumatic disease.

According to a first aspect, the invention relates to a method for preparing a macromolecule produced by bacteria of the Bifidobacterium breve I-2219 strain, deposited according to the Budapest Treaty, on 31 May 1999 at the National Collection of Microorganisms (Collection Nationale de Cultures de Microorganismes—CNCM) held at the Pasteur Institute, 25 rue du Docteur Roux, in Paris.

The said method is characterised in that it comprises the following steps:

i) Seeding and incubation of a Bifidobacterium breve I-2219 strain, under aerobic or anaerobic conditions at a temperature approximately comprised between 30° C. and 40° C., in culture medium with a regulated or non-regulated pH and containing as ingredients a fraction of lactoserum proteins enriched with native (non denatured) lactalbumin and lactose;

ii) Preparation of the said bacteria from the said culture medium after a period of incubation comprised between 16 and 48 hours;

iii) Ultrafiltration on filtration membranes with a cutoff threshold of 2 kDa to 50 kDa, to obtain a concentrated retained fraction;

iv) Macromolecule enrichment by washing with a volume equal to 5 to 25 times the volume of the concentrated retained fraction;

v) Dehydration of the said concentrated enriched retained fraction;

vi) Purification of the macromolecule by running over an anion-exchange resin;

vii) Collection of the eluate with 0.15-0.6M NaCl, preferably 0.3M NaCl, the eluate containing the bacterial macromolecule.

According to various embodiments, the method for the preparation of the said macromolecule of bacterial origin presents the following characteristics, which may be combined as the case may be:
Seeding of the bifidobacteria in the said culture medium is carried out starting from frozen concentrate or a 16 to 24-hr pre-culture, allowing the proliferation of bacteria;
The bacteria are seeded in the said culture medium at a concentration of 10⁵-10¹⁰ colony forming units per ml of medium;
The ingredients of the culture medium are present in the following quantities:
   fraction of lactoserum proteins enriched with native (non denatured) lactalbumin: 0.01 to 1 g/ml of medium;
   lactose: 30-70 9/litre of medium;
The pH of the said culture medium is not regulated;
The pH of the said culture medium is maintained between about 4 and 6 units.

According to a second aspect, the invention relates to a macromolecule of bacterial origin produced by bacteria of the Bifidobacterium breve I-2219 strain prepared by implementing the method of the invention. The tests carried out by the applicant have shown that the said molecule has anti-arthritic activity in mice, in a model of arthritis induced by collagen. This anti-arthritic activity has been evaluated by various parameters, including:
Protection from the arthrogenic effect of collagen II in mice;
Properties of regulation of the intestinal flora and of bacterial translocation.

According to a third aspect, the present invention aims at using the said macromolecule of bacterial origin for the preparation of medicines designed for the prevention or treatment of inflammatory rheumatic disease. The medicines containing the said molecule can be used as an adjuvant to the therapies currently in use for the treatment of inflammatory rheumatic disease (leflunomide, monoclonal antibodies, anti-inflammatory agents) or replace these medicines, the side effects of which are well documented.

The medicines containing the said molecule can be administered per os or by subcutaneous injection; they have a regulatory effect on the intestinal flora and bacterial translocation, and hence, reduce the inflammatory reaction associated with inflammatory rheumatic disease. These effects are in favour of their recommendation both for prevention of rheumatic disease (for patients from families at risk) and for the treatment of various forms of inflammatory rheumatic disease, including rheumatoid polyarthritis and ankylosing spondylitis.

The medicines containing the said molecule of bacterial origin prepared according to the invention can be stored for 3 years at a temperature of −35° C. to +4° C. for liquid forms and up to +40° C. in the case of dry forms. The said medicines do not cause side effects.

The present invention will be better understood upon reading the description to be made in reference to the following examples of embodiment.

### EXAMPLE 1

### Preparation of a Macromolecule Obtained by Bifidobacterium breve Culture

Culture medium containing the following ingredients is prepared:
lactalbumin-enriched preparation corresponding to 0.01-0.05 g of lactalbumin per litre of prepared medium;
0.3 g/l of cysteine chlorohydrate;
2.15 g/l of potassium dihydrogenphosphate;
62.5 g/l of lactose.

The lactalbumin-enriched preparation is achieved by precipitating lactoserum proteins out of solution (for example Vitalarmor alpha 607 distributed by ARMOR PROTEINES) at pH 4.1 for 15 minutes in a water bath at 60° C. The supernatant is then collected after centrifugation.

A first solution is reconstituted with the lactose alone. A second solution is reconstituted with the rest of the ingredients. The two solutions are autoclaved for 30 minutes at 115° C.

Continuous Fermentation at a Regulated pH

The pH of the medium is adjusted to a value of 5.8 once the medium is poured into the fermentator. The culture medium is seeded with 6.67% (v/v) (100 ml of inoculum for 1500 ml of medium) of a 24-hr inoculum containing between 1×10⁶ and 2×10⁸ colony forming units (CFUs) of bifidobacteria per ml of culture medium. The bifidobacteria are cultivated with shaking, without aeration of the medium, at a temperature of 37° C. The pH is maintained at 5.8 by adding 3N sodium hydroxide. Fermentation lasts approximately 20 hours and the density of Bifidobacterium breve at the end of bacterial culture is comprised between 1×10⁹ and 1×10¹⁰ CFUs per ml of culture medium. The growth of bacteria corresponds on average to a 10-fold increase in the bacterial population.

Discontinuous Fermentation at a Non Regulated pH

The pH of the medium is not adjusted when the medium is poured into the fermentator. The culture medium is seeded with 6.67% (v/v) (100 ml of inoculum for 1500 ml of medium) of a 24-hr inoculum containing between 1×10⁶ and 2×10⁸ colony forming units (CFUs) of bifidobacteria per ml of culture medium. The bifidobacteria are cultivated without shaking, without aeration of the medium, at a temperature of 37° C. Fermentation lasts about 20 hours.

In a variation of this embodiment, the seeding of the bifidobacteria in the said culture medium is carried out starting from a frozen concentrate containing 10⁸ to 10¹² CFUs of bifidobacteria per g of concentrate.

Quantitative Aspects

In a fermentation at regulated pH, the yield of product recovered after ultrafiltration is 700-1,000 mg of dry powder per litre of fermented medium. The quantity of proteins, determined by the Lowry method, is 275 mg/g of dry powder. The quantity of sugars, determined by the Dubois method, is 220 mg/g of dry powder.

When the pH is non regulated, the yield of product recovered after ultrafiltration is in the order of 70-170 mg of dry powder per litre of fermented medium. The quantity of proteins, determined by the Lowry method is in the order of 71 mg/g of dry powder. The quantity of sugars, determined by the Dubois method, is 615 mg/g of dry powder.

At the end of the bacterial culture, the bacteria are eliminated by centrifugation for 30 minutes at 8,000 g.

The supernatant is ultrafiltrated at room temperature on an AMICON Proflux M12 device equipped with an AMICON spiral S10Y10-type cartridge with a cutoff threshold of 10 kDa. In the first instance, the supernatant is concentrated 10 times at the most, then washed continuously with 15 litres of osmosed water. At the end of the washing step, the retained fraction is concentrated to a volume of 500 ml and stored in frozen or lyophilised form.

The macromolecule contained in the retained fraction is then purified by running the concentrated solution of retained fraction or a solution of reconstituted retained fraction over an anion exchange resin (fast-flow DEAE-Sepharose distributed by Amersham Biosciences, equilibrated with 50 mM Tris-HCl buffer, pH 8.0; elution is carried out with the same buffer, to which NaCl at a concentration of 0 to 1 M has been added).

The bacterial macromolecule is collected in the fraction eluted by 0.15-0.6M NaCl (preferably 0.3M NaCl). The salt is removed from the fraction, which is then lyophilised. The macromolecule thus produced is made up of oligosaccharides of molecular mass comprised between 5 and 30 kDa and a protein of bacterial origin of molecular mass comprised between 50 and 70 kDa.

### EXAMPLE 2

### Determination of Anti-Rheumatic Properties of the Macromolecule Derived from the Bifidobacterium breve I-2219 Strain

The anti-rheumatic properties of the macromolecule of bacterial origin prepared according to the invention (constituting the purified fraction obtained by the procedure described in example 1) have been evaluated in an experimental model of rheumatoid arthritis, i.e. arthritis induced by collagen II in male DBA/1 mice.

2.1 Protection Against the Arthrogenic Effect of type-II Collagen in Male DBA/1 Mice

The mice used were divided into two groups:
a control group of 15 mice (immunised with collagen II);
a test group of 5 mice (immunised with collagen II).

Two collagen II injections were given at the base of the tail of male DBA/1 mice. The animals were 4-5 weeks-old at the time of the first injection. The booster was given three weeks later. In the weeks that followed, their articulations became swollen. A score of arthritis was established per animal and readings were taken several times a week.

Products administered to tested animals:
control: instead of drinking water, animals from the control group were given the non fermented culture medium treated by 10 kDa ultrafiltration as per the conditions of preparation of the macromolecule of bacterial origin;
macromolecule:
1) Loading dose: 100-150 microlitres per mouse at the beginning of the experiment, then at 10, 16, 28, 38 and 56 days post-immunisation (10 g/l solution expressed as dry weight of product)
2) Maintenance dose: 0.5-1.3 g/l solution (expressed as dry weight of product) given to animals ad libitum instead of drinking water.

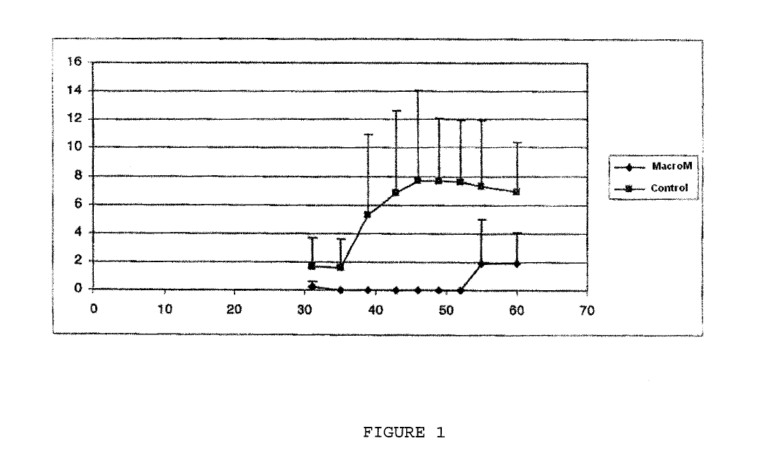

FIG. 1 in the annex to the description illustrates the evolution of the arthritic scores as a function of time for the mice of the control group versus the mice of the test group; It shows that administration of the macromolecule prevents the appearance of arthritic signs, the difference between the two groups (calculated using the ANOVA test) being significant (p<0.001).

2.2 Determination of the Properties of Regulation of the Intestinal Flora and Bacterial Translocation

Other than the two groups of animals immunised with collagen II, animals receiving either the culture medium (10 male DBA/1 mice), or the macromolecule (6 male DBA/1 mice) were included in the study. They were not immunised by collagen II but were distributed among the cages of the immunised animals. They did not develop arthritis.

At the end of the experiment (i.e. 58 to 68 days after the first injection of collagen), the anaerobic flora and the aerobic-anaerobic facultative flora was enumerated in the caecum, Peyer's patch, heart blood, kidneys, spleen, liver and lungs. The animals were sacrificed at least two hours after the last food intake.

Table 1 presents the results concerning the total intestinal flora, expressed as the mean and standard deviation of the log of the number of CFUs/g of faecal matter, where n is the number of mice for each product tested (ANOVA comparison to two different factors).

TABLE 1

| Caecum flora | | | | | |
|---|---|---|---|---|---|
| Mice | Control | | Test | | ANOVA |
| Immunisa- | − | + | − | + | |
| tion | | | | | |
| (collagen) | | | | | |
| N | 10 | 15 | 6 | 5 | |
| Total flora | 8.6 ± 0.8 | 9.3 ± 0.3 | 8.6 ± 0.5 | 8.8 ± 0.9 | 0.037 |

In mice having developed arthritis (immunised control group), the caecum flora is more abundant than in non immunised animals (non immunised control group) (p<0.0469). This bacterial proliferation is not found in immunised animals receiving the macromolecule (immunised test group). The macromolecule therefore regulates the intestinal flora.

Bacterial Translocation

Bacterial translocation of intestinal origin was analysed in the Peyer's patch. The results are presented in Table 2. In mice developing arthritis (immunised control group), the total flora does not increase but its bacterial composition changes. A higher contamination by Gram positive bacilli is observed in immunised control mice (ANOVA p=0.05). This increase in bacilli is not found in the immunised group treated with the macromolecule. The macromolecule therefore contributes to the better regulation of bacterial translocation specifically induced by collagen immunisation.

Furthermore, the macromolecule reduces the passage of bacteria in non immunised animals.

TABLE 2
Bacterial contamination of Peyer's patch

| Mice | Control | | Test | |
|---|---|---|---|---|
| Collagen | − | + | − | + |
| N | 10 | 15 | 6 | 5 |
| Total flora | 4.4 ± 0.8^{a}* | 4.3 ± 0.9 | 3.5 ± 0.6* | 4.0 ± 0.7 |
| Enterococci | 4.0 ± 1.0 | 3.5 ± 1.2 | 2.9 ± 0.9 | 3.5 ± 1.0 |
| | (8) | (13) | (5) | (3) |
| Enterobacteria | 5.5 (1) | 3.3 ± 0.4 | 0 | 3.8 (1) |
| | | (5) | | |
| Gram + bacilli | 3.3 ± 1.1 | 4.1 ± 0.9 | 3.0 ± 0.8 | 3.5 ± 0.6 |
| | (8) | (14) | | |

^{a}log CFUs/PP (log colony forming units/Peyer's patch)
*p < 0.05 (Mann Whitney test)

Bacterial Dissemination

The bacteria having crossed the intestinal barrier are eliminated by macrophages and polynuclear cells in the blood and by macrophages and dendritic cells in internal organs (lungs, liver, spleen, kidneys). The results relative to bacterial dissemination, presented in Table 3, show that the majority of control mice, whether immunised or not, harbour living bacteria in the liver and kidneys.

TABLE 3
Bacterial contamination of internal organs

| Mice | | | | |
|---|---|---|---|---|
| Collagen | − | + | − | + |
| N | 10 | 15 | 6 | 5 |
| Blood | 4^{a} (40)^{b} | 7 (47) | 4 (67) | 1 (20) |
| Kidneys | 8 (80) | 11 (73) | 3 (50) | 3 (60) |
| Spleen | 3 (30) | 7 (47) | 4 (67) | 1 (20) |
| Lungs | 6 (60) | 7 (47) | 2 (33) | 2 (40) |
| Liver | 8 (80) | 12 (80)* | 2 (33) | 1 (20)* |

a: number of mice harbouring microorganisms in the organ under consideration
b: percentage of mice harbouring microorganisms in the organ under consideration
*p=0.029 (Fisher's exact test)

The isolation of live bacteria from the liver is rarer in immunised animals treated with the macromolecule (p=0.029).

The macromolecule therefore allows better regulation of bacterial contamination in the liver.

1: Method for the preparation of a macromolecule produced by bacteria of the Bifidobacterium breve 1-2219 strain, characterised in that it involves the following steps:
i) the seeding and incubation of a Bifidobacterium breve I-2219 strain under aerobic or anaerobic conditions, at a temperature comprised approximately between 30° C. and 40° C., in a culture medium with a regulated or non regulated pH and containing as ingredients a fraction of lactoserum proteins enriched with native (non denatured) lactalbumin and lactose;
ii) the separation of said bacteria from said culture medium after a period of incubation comprised between 16 and 48 hrs;
iii) the ultrafiltration on filtration membranes with a cutoff threshold between 2 kDa and 50 kDa, to obtain a concentrated retained fraction;
iv) macromolecule enrichment by washing with a volume equal to 5 to 25 times the volume of concentrated retained fraction;
v) the dehydration of said concentrated enriched retained fraction;
vi) the purification of the macromolecule by running over an anion exchange resin;
vii) the collection of the fraction eluted with 0.15-0.6M NaCl, preferably 0.3M NaCl, containing the bacterial macromolecule.

2: A method according to claim 1 characterised in that the seeding of the bifidobacteria in said culture medium is carried out starting from a frozen concentrate or a 16 to 24-hr pre-culture.

3: A method according to claim 1 or 2 characterised in that the bacteria are seeded in said culture medium at a density of 10⁵ to 10¹⁰ colony forming units per ml of medium.

4: A method according to claim 1 or 2 characterised in that the ingredients of the culture medium are present in the following quantities:
fraction of lactoserum proteins enriched with native (non denatured) lactalbumin: 0.01 to 1 g/litre of medium;
lactose: 30-70 g/litre of medium.

5: A method according to claim 1 or 2 characterised in that the pH of said culture medium is not regulated.

6: A method according to claim 1 or 2 characterised in that the pH of said culture medium is maintained between about 4 and 6.

7: Preparation based on the bacterial macromolecule obtained by the implementation of the method according to claim 1 or 2, to obtain a product designed to regulate the intestinal flora and its translocation.

8: Preparation based on the bacterial macromolecule obtained by the implementation of the method according to claim 1 or 2 to obtain a pharmaceutical composition designed to treat rheumatoid polyarthritis.

9: Use of the macromolecule of bacterial origin obtained by the implementation of the method according to claim 1 or 2 for the preparation of medicines for the treatment or prevention of inflammatory rheumatic disease.
